(19) United States
(12) Patent Application Publication
(10) Pub. No.: US 2025/0366968 A1
(43) Pub. Date: Dec. 4, 2025

(54) IMPLANTABLE PROSTHESIS WITH SELF-FIXATING GRIPS

(71) Applicant: Davol Inc., Warwick, RI (US)

(72) Inventors: Augustus Felix, Cranston, RI (US); Talia J. D'Ambruoso, San Jose, CA (US); Jeremy Griffin, North Attleboro, MA (US)

(73) Assignee: Davol Inc., Warwick, RI (US)

(21) Appl. No.: 18/877,064

(22) PCT Filed: Jun. 15, 2023
(86) PCT No.: PCT/US2023/025382

§ 371 Date: Dec. 19, 2024

Related U.S. Application Data
(60) Provisional application No. 63/357,099, filed on Jun. 30, 2022.
(60) Provisional application No. 63/357,105, filed on Jun. 30, 2022.

(51) Int. Cl. A61F 2/00

(52) U.S. Cl. CPC A61F 2/0077; A61F 2220/0008; A61F 2220/0016; A61F 2230/0008; A61F 2230/0043; A61F 2230/0069; A61F 2240/001

(57) ABSTRACT

An implantable prosthesis for repairing tissue defects includes a layer of repair fabric and a plurality of microtextured grip segments mounted to the repair fabric. The grip segments include perforations on a tissue facing surface to promote tissue ingrowth.

### RELATED APPLICATIONS

This application claims the benefits of U.S. application No. 63/357,099, filed Jun. 30, 2022 and U.S. application No. 63/357,105, filed Jun. 30, 2022, both of which are hereby incorporated by reference in its entirety.

### FIELD

Disclosed embodiments are related to an implantable prosthesis, and more particularly to a prosthesis for mending defects and weaknesses of soft tissue and muscle walls.

### BACKGROUND

A defect in a muscle or tissue wall, such as a hernia, is commonly repaired with an implantable prosthesis that is configured to cover and/or fill the defect.

For some procedures, an implantable repair fabric, such as a mesh fabric, is sutured, stapled, tacked, or otherwise provisionally anchored in place over, under or within the defect. Tissue integration with the fabric, such as tissue ingrowth into and/or along the mesh fabric, eventually completes the repair.

Various repair fabrics are known and used for repairing soft tissue and muscle wall defects. Examples of implantable fabrics that have been successfully used in soft tissue and muscle wall repair include BARD Soft Mesh, BARD Mesh and VISILEX, available from C.R. Bard. Such fabrics are fabricated from polypropylene monofilaments that are knitted into a mesh having pores or interstices that promote tissue ingrowth and integration with the fabric.

For some procedures, it may be desirable to employ an implantable prosthesis configured to fit the shape of the anatomical region of the defect. In some instances, such a prosthesis can be positioned and maintain its position relative to the defect with little or no provisional fixation. Examples of implantable prostheses that have been successfully used in soft tissue and muscle wall repair include 3DMAX Light Mesh and 3DMAX Mesh, available from C.R. Bard. Such prostheses are fabricated from a mesh fabric formed into a curved, 3-dimensional shape that fits the anatomical shape of the defect region, such as the inguinal anatomy. These prostheses have proven useful and have become established in the practice of muscle or tissue wall repair in the inguinofemoral region.

It is an object of the disclosure to provide a prosthesis for mending defects in soft tissue and muscle walls.

### SUMMARY

An implantable prosthesis is provided that comprises a layer of biologically compatible repair fabric and a plurality of grip segments integrated with the layer of repair fabric. Each of the plurality of grip segments includes a plurality of grips and a plurality of perforations extending therethrough.

An implantable prosthesis is provided that comprises a layer of biologically compatible repair fabric and a plurality of microtextured grip segments integrated with the layer of repair fabric. Each of the plurality of microtextured grip segments includes a plurality of perforations extending therethrough.

An implantable prosthesis is provided that comprises a layer of biologically compatible repair fabric and a plurality of grip segments integrated with the layer of repair fabric. Each of the plurality of grip segments includes a substrate and a plurality of grips extending from a surface of the substrate, wherein each of the plurality of grips includes a first pillar and a plurality of second pillars extending from a surface of the first pillar.

A grip segment is provided for an implantable prosthesis. The grip segment comprises a substrate of biologically compatible material attachable to a layer of repair fabric and a plurality of microstructure grips extending from a surface of the substrate. Each of the plurality of microstructure grips includes a first pillar and a plurality of second pillars extending from a first surface of the first pillar. The grip segment also comprises a plurality of perforations extending through the substrate.

A method is provided for fabricating an implantable prosthesis. The method comprises providing a layer of biologically compatible repair fabric and attaching a plurality of grip segments to a surface of the layer of repair fabric. Each of the grip segments includes a plurality of grips and a plurality of perforations extending therethrough.

An implantable prosthesis is provided that comprises a layer of biologically compatible repair fabric having a preformed three-dimensional configuration with an apex and a rounded ridge extending from the apex to an outer periphery of the layer of repair fabric. The implantable prosthesis also comprises a plurality of grip segments integrated with the layer of biologically compatible repair fabric, wherein a portion of at least one of the plurality of grip segments extends across the rounded ridge.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is an embodiment of an implantable prosthesis according to one embodiment;

FIG. 2 is an embodiment of a layer of repair fabric that may be preformed into a configuration for use as a prosthesis;

FIG. 3A is top view of an embodiment of a grip segment;

FIG. 3B is a top view of an embodiment of a grip segment;

FIG. 4A is a schematic of a perforation of the grip segment of FIG. 3A, according to an embodiment;

FIG. 4B is a schematic of a perforation of the grip segment of FIG. 3B, according to an embodiment;

FIG. 5A is top perspective view of an embodiment of a microtextured film;

FIG. 5B is a top plan view of the microtextured film of FIG. 5A;

FIG. 5C is an enlarged view of section 5C of FIG. 5B;

FIG. 5D is a side view of the microtextured film of FIG. 5A;

FIG. 5E is enlarged view of section 5E of FIG. 5D;

FIG. 5F is a schematic of a microtextured film according to one embodiment contacting a fluid;

FIG. 6 is an embodiment of a prosthetic;

FIG. 7 is an embodiment of a prosthetic;

FIG. 8 is an embodiment of a prosthetic;

FIG. 9 is an embodiment of a prosthetic;

FIG. 10 is an embodiment of a prosthetic;

FIG. 11 is an embodiment of a prosthetic;

FIG. 12 is a side schematic view of a body portion according to one embodiment;

FIG. 13A is an embodiment of a prosthetic; and

FIG. 13B is an embodiment of a prosthetic.

### DETAILED DESCRIPTION

The disclosure is directed to an implantable prosthesis for mending an anatomical defect, and is particularly suitable for mending defects in, and weaknesses of, soft tissue and muscle walls or other anatomical regions. The phrase “mending a defect” includes acts of repairing, augmenting, and/or reconstructing a defect and/or a potential defect. For ease of understanding, and without limiting the scope of the disclosure, the prosthesis is described below particularly in connection with mending a groin defect including, but not limited to, one or more of an indirect inguinal hernia, a direct inguinal hernia, a femoral hernia and/or other weakness or rupture of the groin anatomy. It should be understood, however, that the prosthesis is not so limited and may be employed in other anatomical procedures, as should be apparent to one of skill in the art. For example, and without limitation, the prosthesis may be employed for ventral hernias, chest or abdominal wall reconstruction, or large defects, such as those that may occur in obese patients. The prosthesis may include one or more features, each independently or in combination, contributing to such attributes.

The disclosure is more particularly directed to a prosthesis which includes a repair fabric having a body portion that is configured to cover or extend across the defect opening or weakness when the body portion is placed against the defect. The prosthesis may be in the form of a patch, although the prosthesis may employ other configurations as should be apparent to one of skill in the art. The patch may have a planar or non-planar configuration suitable for a particular procedure employed for mending a defect.

The prosthesis may be configured with a self-grip arrangement having features that help maintain the position of the prosthesis relative to the defect. The self-grip arrangement may reduce, if not eliminate, separation, sliding, twisting, folding and/or other movement, as may be desired, between the prosthesis and adjacent tissue. Such an arrangement may also reduce, if not eliminate, the need for a surgeon to suture, staple, tack, or otherwise provisionally anchor the prosthesis in place pending tissue integration.

The prosthesis may include one or more grip segments integrated with a layer of repair fabric of the prosthesis. In some embodiments, the grip segments may be integrated with the repair fabric by mounting the grip segments to a surface of the body portion that is configured to engage adjacent tissue. In some embodiments, the grip segments may be formed of variously shaped and sized patches of film. More particularly, the grip segment may include a microstructure arrangement on a gripping surface capable of firmly gripping low coefficient of friction materials without causing damage to the material. The grip segments may be configured to grip the tissue, without employing sutures, by placing and/or pressing the prosthesis against the tissue. In some embodiments, the microtextured film may be configured to grip tissue using fluid mechanics. For example, the microstructure texture of the grip may allow fluid to penetrate into the microstructure providing a high solid-fluid adhesion and high contact angle hysteresis.

The grip segments may be arranged on the body portion in any suitable configuration to provide a desired amount of grip as should be apparent to one of skill in the art. For example, and without limitation, the grip segments may be distributed across the body portion in a uniform, non-uniform or random array, and/or any suitable combination of arrays. The grip segments may be distributed across the entire body portion or located at one or more select regions of the body portion. For example, and without limitation, the grip segments may be located at one or more select regions adjacent one or more segments of the outer periphery of the body portion, and/or one or more select regions located within the inner region of the body portion inwardly away from the outer periphery. Each select region may include one or more grip segments arranged in any suitable pattern within the region.

According to one aspect, the grip segments may be fabricated independent of and mounted to the body portion of the prosthesis. In this manner, the grip segments may be formed from a material that is different from the body portion. For example, and without limitation, the grip segments may be formed of a bioabsorbable material, while the body portion may be formed of a non-absorbable material. Such an arrangement may provide the prosthesis with temporary grip properties during the period of tissue integration, while reducing the amount of foreign material that remains present in a patient's body and maintaining long-term strength of the prosthesis. If desired, the body portion may also be formed of a bioabsorbable material which may be absorbed at a slower rate than the grip segment material.

Independent fabrication of the grip segments may also provide flexibility for configuring the prosthesis. For example, and without limitation, the prosthesis may include grip segments having the same or different grip segment configurations and/or arrangements depending on a particular application of the prosthesis. For example, and without limitation, the prosthesis may include grip segments having the same shape, but mounted in different orientations relative to each other on the body portion. The prosthesis may include grip segments with one or more different shapes in one or more regions of the body portion. In this manner, the prosthesis may be provided with various grip characteristics based on the particular orientations and/or shapes of the grip segments individually and as a whole.

In some embodiments, the grip segments may include a plurality of perforations to allow tissue ingrowth into and through the grip segments. The perforations may be configured in various patterns, sizes, and shapes depending on the desired amount of tissue ingrowth, while maintaining structural integrity of the grip segments. For example, perforations may be distributed across the grip segments in a uniform, non-uniform or random array, and/or any suitable combination of arrays. The perforations may be distributed across the entire grip segment or located at one or more select regions of the grip segment. Each select region may include one or more perforations arranged in any suitable pattern within the region.

In some embodiments, the grip segments may be integrated with a body portion of a prosthesis by securing the grip segments to the body portion using one or more stitches. In some embodiments, attaching the grip segments to the body portion may include a sewing application along the periphery of the grip segment with non-resorbable, such as polypropylene or ePTFE, or resorbable monofilament. In some embodiments, the grip segments may be attached to the body portion using adhesives, bonding, ultrasonic or heat welding, or overmolding. In some embodiments, the grip segments may include one or more protrusions that extend from a surface of the grip segment adjacent the body portion. In a non-limiting example, the protrusions may include a hook or other barbed structure configured to at least partially insert into to a surface of the body portion when the grip is pressed against the body portion to secure the grip segment to the body portion.

The prosthesis may be used for mending soft tissue and muscle wall defects using various surgical techniques, including open, laparoscopic, hybrid (e.g., Kugel procedure), and robotic techniques. During open procedures, the prosthesis may be placed through a relatively large incision made in the abdominal wall and layers of tissue and then the defect is filled or covered with the repair fabric. During laparoscopic and hybrid procedures, the prosthesis may be collapsed, such as by rolling or folding, into a reduced configuration for entry into a subject, either directly through a comparatively smaller incision or through a slender laparoscopic cannula that is placed through the incision. The prosthesis may have particular application with robotic procedures in which placement of the prosthesis is achieved using surgical robotic tools which may involve passage of the prosthesis through a relatively small cannula (e.g., 8 mm) as compared to a cannula (e.g., 10-12 mm) typically employed for more conventional laparoscopic techniques.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

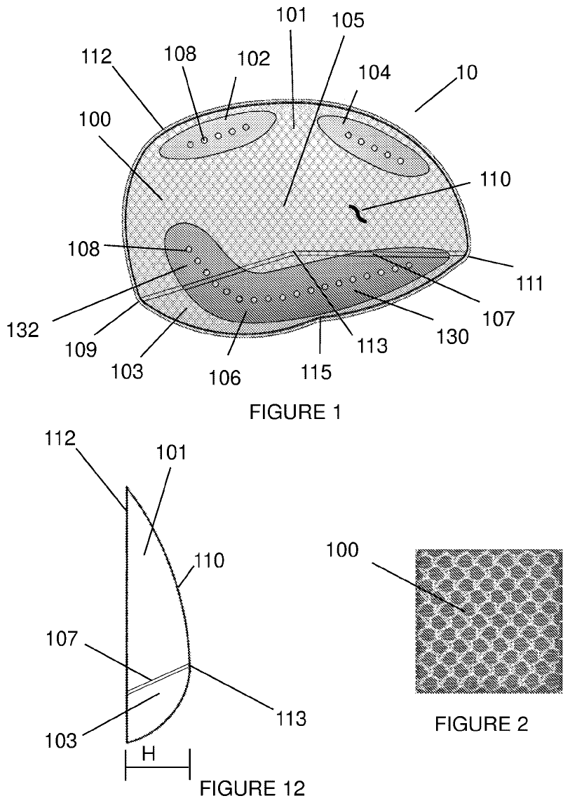

FIG. 1 illustrates one embodiment of a prosthesis for mending tissue and muscle wall defects, such as a hernia defect. The prosthesis includes a repair fabric of implantable, biologically compatible material. In one embodiment, the repair fabric may comprise a mesh fabric that is relatively flexible, thin and light weight and meets the performance and physical characteristics for mending soft tissue and muscle wall defects. The prosthesis 10 may include a body portion 100 configured with a size and/or shape suitable to cover or extend across the defect opening or weakness when the body portion is placed against the defect. The body portion 100 may be a preformed, non-planar patch with a 3-dimensional curved shape. The body portion may include a first region 101 and a second region 103 joined at a rounded ridge 107 that extends from a first outer edge 109 and a second outer edge 111 of the body portion. The rounded ridge 107 may extend from the first and second outer edges 109, 111 in a direction towards the first region 101 and intersect at an angle at an apex 113 arranged near a center region 105 of the body portion between the first and second regions 101, 103. The apex 113 may form the highest point of the body portion relative to a plane formed by the peripheral edge 112 (see also FIG. 12). In one embodiment, the height H of the prosthesis from a plane defined by the peripheral edge 112 and the apex 113 is approximately 21 mm. In some embodiments, the height H of the prosthesis defined by a peripheral edge 112 and the apex 113 is approximately 0.5 inches, 0.7 inches, 0.9 inches, or 1.1 inches. In some embodiments, the apex 113 may be positioned from the left peripheral edge at a distance of approximately 2.75 inches, 2.8 inches, 3.0 inches, 3.15 inches, 2.25 inches, or 3.33 inches. In some embodiments, the apex 113 may be positioned from a bottom peripheral edge, defining the second region of the body portion, at a distance of approximately 1.25 inches, 1.42 inches, 1.5 inches, 1.69 inches, 1.72 inches, 1.75 inches, or 2 inches.

As shown in FIG. 12, the first and second regions 101, 103 may have substantially spherical shapes to enhance conformance to a particular anatomical shape. In one illustrative embodiment, the radius of curvature of the first region is less than the radius of curvature of the second region to form a surface in the second region that has a steeper incline relative to the first region. Returning to FIG. 1, the second region may be shaped to form a depression 115 in the surface of the body portion that is configured, for example, to receive the external iliac vessels when the prosthesis is employed for inguinal hernia repair. The depression extends inwardly from the peripheral edge 112 between the first and second outer edges 109, 111 toward the apex 113.

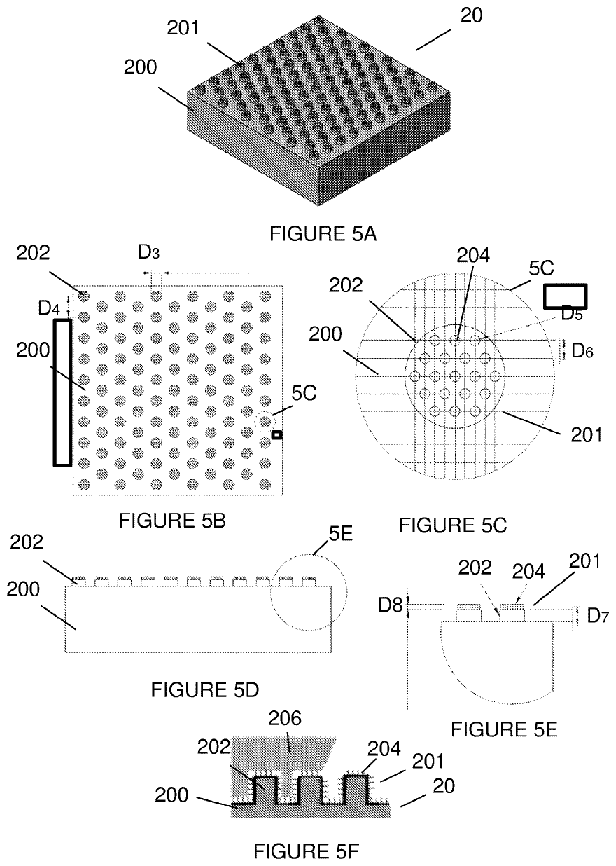

As shown in FIG. 1, the prosthesis 10 may also include one or more grip segments 102, 104, 106 mounted to a surface 110 of the body portion 100 to provide a self-grip arrangement for maintaining the position of the prosthesis relative to the defect. The grip segments may be configured to use fluid mechanics to grip and engage adjacent tissue when the prosthesis is placed and/or pressed against it. For example, the grip segments may include a substrate with a plurality of microstructures (i.e., grips) extending from the substate in a micropattern design, forming a microtextured surface (see FIGS. 5A-5F). The microtextured surface of the grip segment may allow fluid to penetrate between the microstructure grips providing a high solid-fluid adhesion and high contact angle hysteresis. As a result, the grip segments may be able to grip wet tissue while minimizing damage to the tissue.

In some embodiments, the grip segments 102, 104, 106 may be located at one or more grip regions of the body portion 100. Such an arrangement may be suitable for placing grip segments in selected regions of the body portion 100 to accommodate a particular anatomical region. For example, it may be desirable to avoid providing grip segments on regions of the body portion 100 that may potentially contact vessels, nerves, or other portions of the anatomy at the defect site as should be apparent to one of skill in the art. The location of the grip segments may facilitate rolling and/or folding of the prosthesis to deliver to the site of the soft tissue repair. In some embodiments, the grip segments may include one or more perforations 108 to allow tissue ingrowth after implantation into and through the grip segments and into the body portion 100.

As shown in FIG. 1, according to some embodiments, the prosthesis 10 includes first and second grip segments 102, 104 positioned near the peripheral edge 112 on a first region 101 of the body portion 100. The grip segments 102, 104 may be arranged such that each segment is positioned on either side of the apex 113. The grip segments 102, 104 may be configured with an elliptical shape and may or may not be the same shape or size. A third grip segment 106 may be positioned substantially on a second region 103 of the body portion 100 near the peripheral edge 112. The third grip segment 106 may have an L-shape configuration and a first portion 130 of the grip segment may extend at least partially along the peripheral edge 112 of the second region 103 and over the rounded ridge 107 into the first region. A second portion 132 of the grip segment 106 may extend upwards along the peripheral edge 112 over the rounded ridge 107 into the first region 101.

As shown in FIG. 1, the grip segments may be positioned on the body portion 100 proximate to the peripheral edge 112 such that a center region 105 of the prosthesis is free of grip segments. The grip segments 102, 104, 106 may be placed near the peripheral edge 112 such that a user (e.g., surgeon, physician, etc.) may see through the prosthesis when it is being implanted in a patient over a defect. In addition, such a placement may reduce or prevent the grip segments from contacting nerves or other potentially sensitive regions when implanted, which may cause pain for the patient. For example, the L-shape of the third grip segment 106 may be positioned such that it avoids nerves, blood vessels, or other tissue when implanted at the defect site.

The size, shape, and location of the grip segments may be configured and arranged to balance the gripping force, tissue ingrowth potential, and flexibility of the prosthesis. For example, the grip segments may increase gripping force while reducing the exposed mesh surface required for rapid tissue ingrowth. In this manner, the grip segments may act as a barrier to rapid tissue ingrowth until the grip segment breaks down. Providing the one or more perforations in the grip segments may allow rapid tissue ingrowth prior to the grip segment breaking down. The grip segments may also reduce the flexibility of the prosthesis, which may need to be rolled up to fit into a narrow cannular or trocar to reach a target site during a procedure. The configuration and arrangement of the grip segments 102, 104, 106 illustrated in FIG. 1 may allow the prosthesis to maintain sufficient flexibility while still providing sufficient surface area with gripping portions to securely adhere to tissue.

The number, shapes, sizes, and position of the grip segments 102, 104, 106 may vary according to particular procedures, and those shown in the figures are merely non-limiting examples, as the disclosure is not so limited. For example, the grip segments may take on any symmetrical or asymmetrical shape, including but not limited to, circular, polygonal, arched, elliptical, or any freeform shape. In some embodiments, the grip segments may be positioned along the periphery of the prosthesis, but in some embodiments the grip segments may be positioned near center region of the prosthesis or may be arranged to cover a significant surface 110 of the prosthesis, depending on need. Alternatively, fewer or more than three grip segments may be used. For example, four elliptically shaped grip segments may be positioned around the periphery of a body portion, or two L-shaped grip segments may be positioned opposite each other in the first and second regions 101, 103. Any suitable number of grip segments may be used to achieve a desired gripping force and flexibility.

In some embodiments, the body portion 100 may be a preformed, non-planar patch with a 3-dimensional curved shape. In one embodiment, the body portion 100 may have a shape corresponding to the 3DMAX MID, 3DMAX Light Mesh or 3DMAX Mesh, available from Davol, and described in one or more of U.S. Pat. Nos. 6,723,133, 6,740,122 and 6,740,122. In this manner, the prosthesis may be particularly suited for fitting and mending defects to the inguinal anatomy. However, it is to be understood that the prosthesis may employ other configurations as should be apparent to one of skill in the art. For example, and without limitation, the body portion may have a planar or other non-planar configurations suitable for a particular procedure employed for mending a defect. Moreover, the prosthesis may be provided as a planar sheet of self-gripping repair fabric that may be selectively trimmed by a surgeon to any desired size and shape for the particular procedure.

In some embodiments, a length of the body portion may be greater than or equal to 5.25 inches, 5.5 inches, 5.75 inches, 6.0 inches, 6.25 inches, and/or any other appropriate length. In some embodiments, the length may be less than or equal to 6.25 inches, 6.5 inches, 6.75 inches, 7 inches, and/or any other appropriate width. Combinations of the foregoing are contemplated including, for example, a length that is between or equal to 5.5 inches and 6.65 inches, and/or any other appropriate combination of the foregoing. In some embodiments, a width of the body portion may be greater than or equal to 3.25 inches, 3.5 inches, 3.75 inches, 4.0 inches, 4.25 inches, and/or any other appropriate width. In some embodiments, the width may be less than or equal to 4.5 inches, 4.75 inches, 5.0 inches, 5.25 inches, and/or any other appropriate width. Combinations of the foregoing are contemplated including, for example, a width that is between or equal to 3.5 and 4.85 inches, and/or any other appropriate combination of the foregoing. While specific ranges for the length and width are provided above, it should be understood that ranges both greater than and less than those noted above are also contemplated as the disclosure is not so limited.

FIG. 2 illustrates an embodiment of a layer of repair fabric that may be preformed into a configuration for use as a prosthesis or may be selectively trimmed into a desirable configuration for use as a prosthesis or a portion of a prosthesis. The repair fabric may employ a knit construction that provides openings or pores to allow tissue infiltration to incorporate the prosthesis. When implanted, the mesh promotes rapid tissue or muscle ingrowth into and around the mesh structure. Examples of surgical materials which may be utilized for the layers and are suitable for tissue or muscle reinforcement and defect correction include, but are limited to, BARD Mesh (available from Davol, Inc.), BARD Soft Mesh (available from Davol, Inc.), SOFT TISSUE PATCH (microporous ePTFE—available from W.L. Gore & Associates, Inc.); SURGIPRO (available from US Surgical, Inc.); TRELEX (available from Meadox Medical); PROLENE and MERSILENE (available from Ethicon, Inc.); and other mesh materials (e.g., available from Atrium Medical Corporation). Absorbable or resorbable materials, including PHASIX Mesh (available from Davol, Inc.), polyglactin (VICRYL—available from Ethicon, Inc.) and polyglycolic acid (DEXON—available from US Surgical, Inc.), may be suitable for applications involving temporary correction of tissue or muscle defects. Collagen materials such as COOK SURGISIS, available from Cook Biomedical, Inc. may also be used. It also is contemplated that the mesh fabric may be formed from multifilament yarns and that any suitable method, such as knitting, weaving, braiding, molding and the like, may be employed to form the mesh material.

The repair fabric may also have sufficient flexibility to promote an easy reduction in size for entry into the subject. In this manner, the flexible fabric may be collapsed into a slender configuration, such as a roll, which can be supported in, and advanced through, a narrow laparoscopic cannula for use in a laparoscopic or robotic procedures.

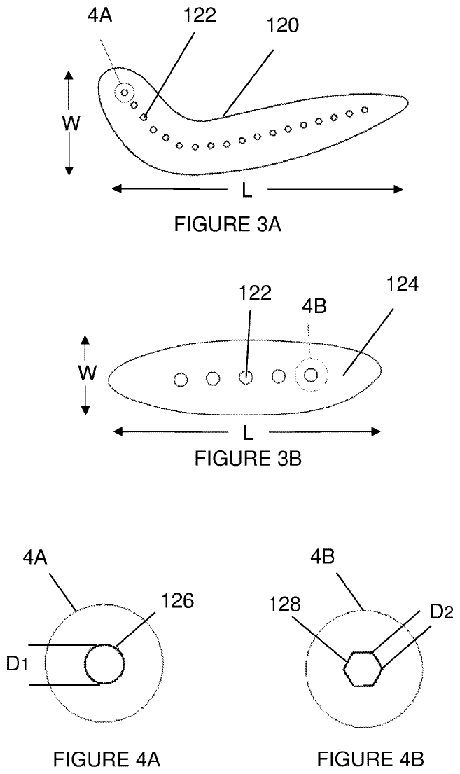

FIGS. 3A-3B illustrate embodiments of an L-shaped grip segment 120 and an elliptical-shaped grip segment 124, respectively. One or more of the L-shaped grip segment 120 and/or elliptical-shaped grip segment 124 may be positioned on a surface of a body portion of a prosthesis.

In some embodiments, a length L of the L-shaped grip segment 120 may be approximately 5.0 inches, 5.25 inches, 5.5 inches, 5.75 inches, 6.0 inches, and/or any other appropriate length. In some embodiments, the length L of the shaped grip segment 120 is approximately 5.4 inches. In some embodiments, a width W of the shaped grip segment 120 may be approximately 1.5 inches, 1.75 inches, 2.0 inches, 2.25 inches, and/or any other appropriate width. In some embodiments, the width W is 2.1 inches. In some embodiments, a thickness of the shaped grip segment 120 may be approximately 155 μm, 165 μm, 175 μm, and/or any other appropriate thickness. Combinations of the foregoing are contemplated including, for example, a length that is between or equal to 5.25 and 5.5 inches, a width that is between 2.0 inches and 2.2 inches, and/or any other appropriate combination of the foregoing.

In some embodiments, a length L of the elliptical-shaped grip segment 124 may be approximately 2.25 inches, 2.5 inches, 2.75 inches, 3.0 inches, and/or any other appropriate length. In some embodiments, the length L of the elliptical-shaped grip segment 124 is approximately 2.7 inches. In some embodiments, a width W of the elliptical-shaped grip segment 124 may be approximately 0.5 inches, 0.75 inches, 1.0 inch, 1.25 inches, and/or any other appropriate width. In some embodiments, the width W of the elliptical-shaped grip segment 124 is 1.0 inch. In some embodiments, a thickness of the elliptical-shaped grip segment 124 may be approximately 150 μm, 155 μm, 165 μm, 175 μm, and/or any other appropriate thickness. Combinations of the foregoing are contemplated including, for example, a length that is between or equal to 2.5 and 2.7 inches, a width that is between 1.8 inches and 1.1 inches, and/or any other appropriate combination of the foregoing. While specific ranges for the dimensions of the grip segments are provided above, it should be understood that ranges both greater than and less than those noted above are also contemplated as the disclosure is not so limited.

As shown in FIGS. 3A-3B, in some embodiments, the grip segments 120, 124 may include a plurality of perforations to allow tissue infiltration to incorporate the prosthesis. When implanted, the porous grip segment promotes rapid tissue or muscle ingrowth into and around the grip segment and body portion. The perforation 122 may extend through the entire thickness of the grip segment or may extend only partially therethrough. The perforations may be included in various patterns on the grip segments. For example, in the embodiment of FIG. 3A, the L-shaped grip segment 120 includes a single array of 18 perforations along a length of the grip segment. In the embodiment of FIG. 3B, the elliptical-shaped grip segment 124 include a single array of 5 perforations along its length. The perforations may be arranged in a uniform, non-uniform or random array, and/or any suitable combination of arrays. The perforations may also be distributed across the entire grip segment.

The perforations provided in the grip segments may be various shapes and sizes to optimize tissue ingrowth and maintain component integrity for insertion of the prosthetic. In some embodiments, the perforations may occupy greater than or equal to approximately 4%, 6%, 8%, 10%, or 12% of the grip segment surface. In some embodiments, the perforations may occupy less than or equal to approximately 25%, 22%, 20%, or 17% of the grip segment surface. Any suitable combinations of the above-referenced ranges are also possible. In one embodiment, the perforations may occupy greater than or equal to 6% and less than or equal to 20% of the grip segment surface.

FIGS. 4A-4B show enlarged views of perforations from sections 4A and 4B in FIGS. 3A-3B. As shown in FIG. 4A, the perforation 126 may be configured with a round shape. The perforation 126 may have a diameter D1 that is approximately 0.003 inches, 0.05 inches, 0.075 inches, 0.100 inches, 0.125 inches, and/or any other appropriate diameter. As shown in in FIG. 4B, the perforation 128 may be configured with a hexagonal shape. The perforation 128 may have a segment distance D2 of approximately 0.025 inches, 0.050 inches, 0.075 inches, and/or any other appropriate distance. Although only two perforations are described, it should be noted that any perforation on a grip segment may be any shape or size depending on the particular need. It is also to be appreciated that a grip segment may employ a combination of perforations having different configurations including, but not limited to, different shapes and/or sizes.

It is to be appreciated that any suitable grip segment arrangement may be provided on the prosthesis to provide a desired amount of grip as should be apparent to one of skill in the art. In some embodiments the prosthesis may include grip segments having the same or different grip configurations and/or arrangements depending on a particular application of the prosthesis. For example, and without limitation, the prosthesis may include grip segments having the same shape, but mounted in different orientations relative to each other on the body portion. The prosthesis may include grip segments with one or more different shapes in one or more regions of the body portion. The grip segments may include various patterns of perforations to facilitate desired levels of tissue ingrowth while maintaining structural integrity. In this manner, the prosthesis may be provided with various grip characteristics for a particular application of the prosthesis based on the configurations and/or arrangements of the grip segments.

The grip segments may be fabricated independent of and mounted to the body portion of the prosthesis (e.g., via adhesives, molding, bonding, sewing, etc.). Independent fabrication of the grip segments may provide flexibility for configuring the prosthesis. In one embodiment, the grip segments may be formed of a bioabsorbable material, while the body portion may be formed of a non-absorbable material. If desired, the body portion may also be formed of a bioabsorbable material which may be absorbed at a slower rate than the grip segment material. Such an arrangement may provide the prosthesis with temporary grip properties during the period of tissue integration, while reducing the amount of foreign material that remains present in a patient's body and maintaining long-term strength of the prosthesis.

In some embodiments, the grip segments may be composed of one or more layers of film. FIGS. 5A-5E show an embodiment of grip segment formed out of a microtextured film. The microtextured film may be cut to form different shaped grip segments, including but not limited to L-shaped and elliptical shaped grip segments. A microtextured film includes a film having a micropattern of microstructures on a surface. As shown in FIG. 5A, the microtextured film 20 includes a substrate 200 and a plurality of microstructure grips 201 on a surface of the substrate. In one embodiment, the film 20 may be single-sided with grips 201 on one surface of the substate, as shown, but the disclosure is not so limited. In one embodiment, the film 20 may be double-sided with grips provided on and protruding from two opposite surfaces of the substrate. Such an arrangement may allow either side of the grip segment to be positioned toward the body portion with the opposite side facing away from the body portion which may facilitate fabrication of the prosthesis. In a non-limiting example, a non-symmetrical grip segment (e.g., an L-shaped grip segment) made of double-sided film may be attached to either a left- or right-handed prosthesis by flipping the grip segment over (i.e., attaching a first surface of the grip segment to a right-handed prosthesis and attaching a second, opposite surface to a left-handed prostheses).

In some embodiments, the grips 201 may be arranged on the substrate 200 in a pattern, such as a triangular lattice shown in FIGS. 5A-5B, but the disclosure is not so limited, and the grips 201 may be arranged on the substrate in any pattern, including but not limited to, a hexagonal, honeycomb, or square lattice. In some embodiments the grips 201 may be positioned on the substrate in a random array.

As shown in FIGS. 5B-5F, each of the plurality of grips 201 may include a first pillar 202 that extends from the surface of the substrate 200 and a plurality of second pillars 204 that extend from a surface of the first pillar 202. The second pillars 204 may be positioned on a top surface of the first pillars 202, as shown in FIGS. 5B-5D, or the second pillars 204 may be positioned on top and/or side surfaces of the first pillars 202 (see FIG. 5F). The second pillars 204 may be arranged on the first pillars 202 in any pattern, including but not limited to a triangle lattice, as shown in FIG. 5C.

In some embodiments, the first pillars 202 may be cylindrical in shape. In some embodiments, the first pillars 202 may have a diameter D3 of approximately 0.09 inches to 0.11 inches and may be spaced apart in a row at a pitch D4 of approximately 0.18 inches to 0.22 inches. The first pillars 202 may have a height D7 of approximately 0.045 inches to 0.055 inches. The first pillars 202 may have a shaft angle of approximately 3° to 7°.

In some embodiments, the second pillars 204 may be cylindrical in shape and extend from a surface of the first pillar 202 in a substantially perpendicular direction. In some embodiments, the second pillars 204 may have a diameter D5 of approximately 0.009 inches to 0.011 inches and may be spaced apart in a row at a pitch D6 of approximately 0.018 inches to 0.022 inches. The second pillars 204 may have a height D8 of approximately 0.018 inches to 0.022 inches. Other cross-sectional shapes (e.g., square, elliptical, orthogonal, etc.), dimensions, and patterns for the first and second pillars are also contemplated, as the disclosure is not so limited. It should be noted that the second pillars 204 may not always extend at an angle perpendicular to a surface of the pillar 202 from which it extends.

In some embodiments, the grip segments may be made from PURASORB® PLDL 7038, a GMP grade copolymer of L-lactide and DL-lactide in a 70/30 molar ratio and with an inherent viscosity midpoint of 3.8 dl/g.

FIG. 5F is a schematic of a portion of a microtextured film 20 contacting a fluid 206. In some embodiments, the second pillars 204 may draw in the fluid between the first pillars of the film 20 such that the fluid penetrates into the grip microstructure between the first pillars 202. This may create a superhydrophobic and hydrophilic surface, resulting in solid-fluid adhesion and therefore high contact angle hysteresis. As a result, grips 201 of the microtextured film 20 may grip wet substrates, including tissue, without damaging the tissue.

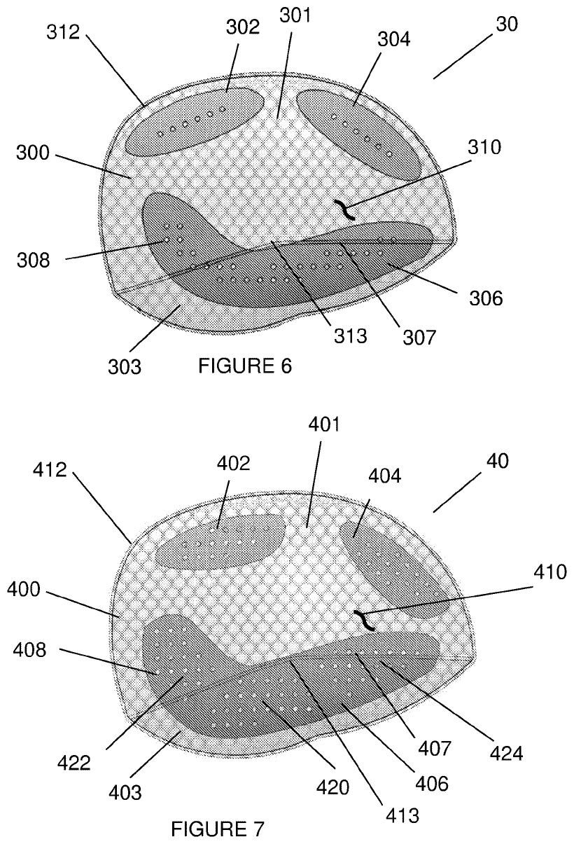

FIGS. 6-10 show embodiments of prostheses with various grip segment and perforation configurations and arrangements. The number, sizes, shapes and configurations of the grip segments may be adjusted depending on application and desired use.

FIG. 6 shows an embodiment of a prosthetic 30 with three grip segments arranged on a surface 310 of a body portion 300. The body portion 300 includes first and second grip segments 302, 304 having an elliptical configuration arranged near a peripheral edge 312 in a first region 301 of the body portion 300. The body portion 300 also includes a third grip segment 306 having an L-shaped configuration arranged near the peripheral edge 312 of the second region 303 and extending over a rounded ridge 307 into the first region 301. The grip segment may be arranged below the apex 313 of the body portion such that the grip segment does not cover the apex. In some embodiments, a grip segment may be arranged to cover the apex.

Each of the grip segments 302, 304, 306 may include a plurality of perforations 308. As illustrated, the perforations 308 for the first and second grip segments 302, 304 may be provided in a linear arrangement along a length of the grip segments. The pattern of perforations for the third grip segment may include multiple rows of perforations 308 along a length of the grip segment. The pattern may include a bottom row of perforations and consecutive rows of perforations stepped outward in a V formation such that the perforations extend along each end of the grip segment in a grid-like fashion. The rows of perforations may be arranged at an angle to a direction along a length of the grip segment. Such a grid-like pattern may be for ease of manufacturing or may facilitate tissue ingrowth in desired locations while maintaining structural integrity of the grip segment and sufficient grip force. However, it is to be appreciated that the grip segments may employ any suitable arrangement of perforations as should be apparent to one of skill in the art.

FIG. 7 shows an embodiment of a prosthetic 30 with three grip segments arranged on a surface 410 of a body portion 300. The body portion 400 includes first and second grip segments 402, 404 arranged near a peripheral edge 412 in a first region 401 of the body portion 400. The body portion also includes a third L-shaped grip segment 406 arranged near the peripheral edge 412 of the second region 403 and extending over a rounded ridge 407 into the first region 401. The first and second grip segments 402, 404 may have an asymmetrical elliptical shape compared to grip segments 302, 303 of FIG. 6 and the third grip segment 406 may have a larger surface area and a larger number of perforations 408 than grip segment 306. Grip segment 404 may be positioned such that it is slightly rotated clockwise around the peripheral edge 412 closer to grip segment 406 than to grip segment 402. Grip 406 may be positioned such that it covers the apex 413.

Each grip segment 402, 404, 406 includes multiple rows of perforations 408. In some embodiments, the first and second grip segments 402, 404 include fifteen (15) perforations aligned in three (3) rows along a length of the grip segment. The larger grip segment 406 includes three (3) separate groups of perforations 408: a center region 420 and two end regions 422, 424. The groups of perforations may be arranged in a grid-like fashion, as shown, with equal sizing and spacing. The distance between adjacent perforations of different groups may be smaller or larger than a distance between perforations of the same group. Alternatively, the perforations may be in a less structured arrangement within each group and may include unequal or random spacing and/or sizes.

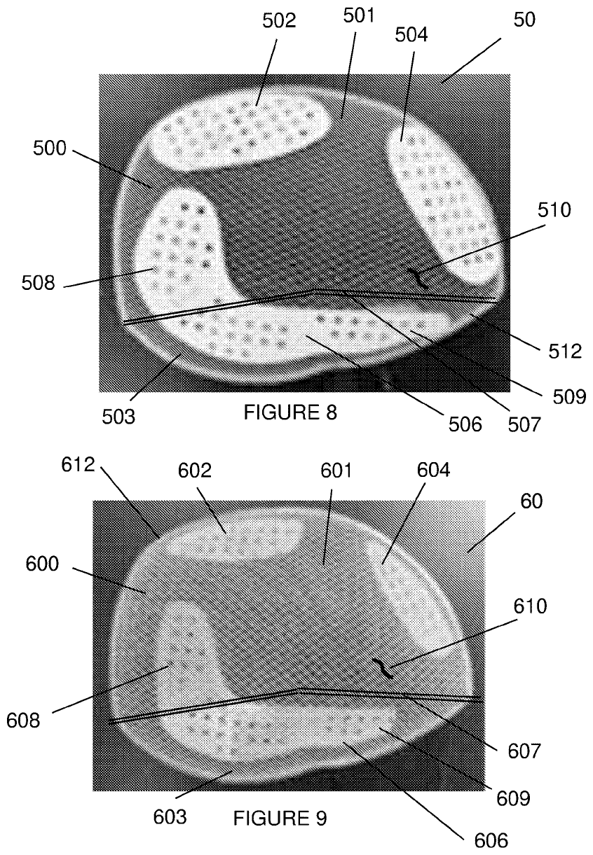

FIG. 8 shows an embodiment of prosthesis 50 having with three grip segments arranged on a surface 510 of a body portion 500. The body portion 300 includes first and second grip segments 502, 504 arranged near a peripheral edge 512 in a first region 501 of the body portion 300. The second grip 504 may be arranged near a side of the peripheral edge 512 near the rounded ridge 517. The first and second grip segments 502, 504 may be larger than the first and second grip segments in FIGS. 2-7. The first and second grip segments 502, 504 may include a pattern of perforations 508 that occupy a significant portion of a surface area of the grip segments. The perforations 508 may be arranged in a grid-like fashion, as shown, with equal spacing.

The body portion 500 also includes a third grip segment 506 arranged near the peripheral edge 512 of the second region 503 and extending upward over a rounded ridge 507 into the first region 501. The third grip segment 506 may be shaped such that one end portion has a larger width than an opposite end portion. The third grip segment 506 may have equally spaced and sized perforations 508 positioned in three (3) separate regions on the grip segment.

FIG. 9 shows an embodiment of prosthesis 60 with three grip segments 602, 604, 606 including a plurality of perforations 608 arranged on a surface 610 of a body portion 600. The body portion 600 includes first and second grip segments 602, 604 arranged near a peripheral edge 612 in a first region 601 of the body portion 600. The body portion 600 also includes a third grip segment 606 arranged near the peripheral edge 612 of the second region 603 and extending upward over a rounded ridge 607 into the first region 601. The third grip segment 606 may be sized with a shorter length relative to the third grip segments of FIG. 8. Comparing FIGS. 8 and 9, the grip segment 506 of FIG. 8 includes a relatively longer end portion 509 that extends to the peripheral edge 512 of the body portion, whereas the grip segment 606 of FIG. 9 has a relatively shorter end portion 609 that terminates prior to the periphery.

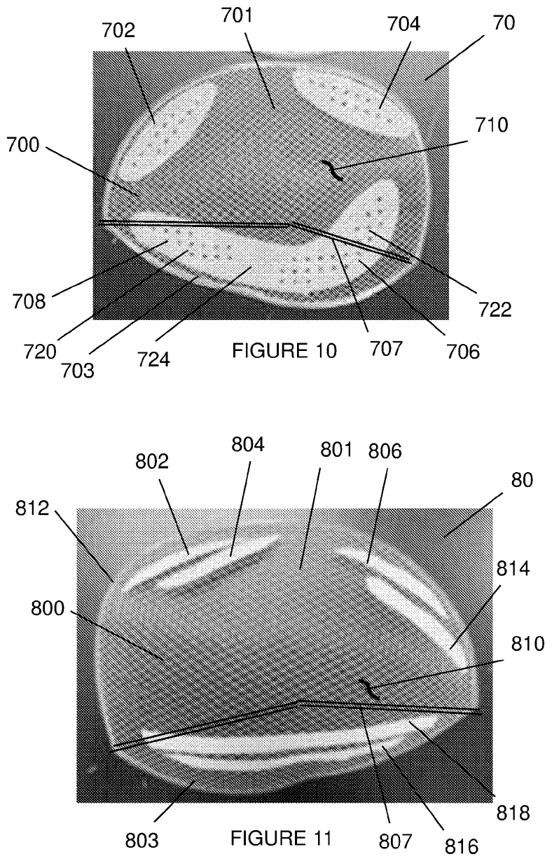

As shown in FIG. 10, a prosthetic 70 includes three grip segments arranged on a surface 710 of a body portion 700. The body portion 700 includes first and second grip segments with twelve (12) perforations 708 arranged in two offset rows of six perforations each. The body portion 700 also includes a third L-shaped grip segment 706 with two (2) groups 720, 722 of perforations 708 separated by a midportion 724 of the grip segment with no perforations. The third L-shaped grip portion 706 may be arranged in the second region 703 of the body portion near the periphery 712, and a portion of the grip portion 706 may extend over the rounded ridge 707 into the first region 701.

In some embodiments, a prosthetic may include any number of grip segments. For example, as shown in FIG. 11, a prosthetic 80 may include four (4) grip segments 802, 804, 806, 814 arranged on a surface 810 of a body portion 800 near a peripheral edge 812 in a first region 801. The body portion may also include two (2) grip segments 816, 818 arranged in the second region 803 of the body portion near the periphery. A portion of one of the grip segments 816, 818 may extend over the rounded ridge 807 into the first region 801. The grip segments may be positioned in pairs with a small amount of mesh of the body portion 800 exposed between a length of each grip segment pair. This may provide more flexibility of the prosthesis and minimize bulk of the prosthesis to allow insertion into a trocar. In some embodiments, the prosthesis may be collapsed to fit within an 8 mm trocar.

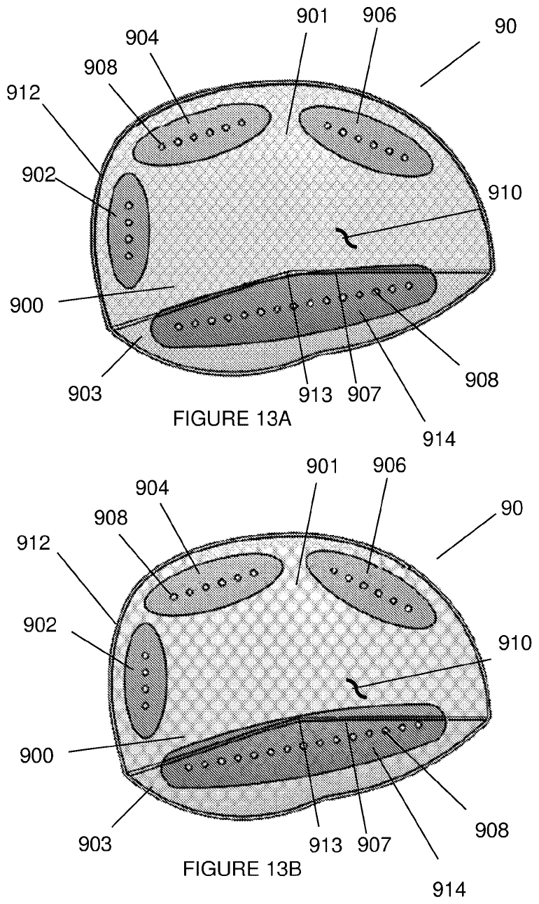

In one embodiment as shown in FIGS. 13A-13B, a prosthesis 90 may include four grip segments 902, 904, 906, 914 arranged on a surface 910 of a body portion 900. Each grip segment may include a plurality of perforations 908. The body portion includes three grip segments 902, 904, 906 that may have an elliptical shape and may be arranged near a first peripheral edge 912 in a first region 901 of the body portion 900. As illustrated, the first region may be located on a first side of a rounded ridge 907. The first grip segment 902 may have a smaller size, such as a shorter length and/or width, relative to the second and third grip segments 904, 906. The first grip segment 902 may be arranged along a segment of the first peripheral edge and the second and third grip segments 904, 906 may be arranged along adjacent segments of the first peripheral edge. The fourth grip segment 914 may be arranged in a second region 903 of the body portion 900 located on a second side of the rounded ridge 907 and extend along a second peripheral edge 912. In some embodiments, the fourth grip segment 914 may be arranged entirely in the second region 903, or a portion of the fourth segment 914 may contact or partially extend over the rounded ridge 907 into the first region 901. As shown in FIG. 13A, the fourth grip segment 914 may contact the rounded ridge 907 at one side of the grip segment but not extend over the apex 913. As shown in FIG. 13B, the fourth grip segment 914 may extend over the rounded ridge 907 and the apex 913 on one side of the grip segment. The fourth grip segment 914 may be elliptical in shape and may be larger, such as sized with a longer length and/or width, relative to the second and third grip segments 904, 906.

In some embodiments, the first grip segment 902 may have a length of approximately 1.25 inches, 1.50 inches, 1.75 inches, 1.90 inches, or 2.0 inches and a width of approximately 0.50 inches, 0.70 inches, or 0.80 inches. In some embodiments, the first grip segment 902 may have a length of approximately 1.89 inches and a width of approximately 0.689 inches. In some embodiments, the first grip segment 902 may have a length of approximately 1.51 inches and a width of approximately 0.614 inches. In some embodiments, the second and third grips 904, 906 may have a length of approximately 1.75 inches, 2.0 inches, 2.25 inches, or 2.5 inches and a width of approximately 0.50 inches, 0.60 inches, 0.70 inches, 0.80 inches, or 0.90 inches. In some embodiments, the second and third grip segments 904, 906 may have a length of approximately 2.362 inches and a width of approximately 0.787 inches. In some embodiments, the second and third grip segments 904, 906 may have a length of approximately 2.047 inches and a width of approximately 0.632 inches. In some embodiments, the fourth grip segment 914 may have a length of approximately 3.75 inches, 4.0 inches, 4.50 inches, inches, 4.75 inches, or 5.0 inches and a width of approximately 0.75 inches, 1.0 inches, or 1.25 inches. In some embodiments, the fourth grip segment 914 may have a length of approximately 4.762 inches and a width of approximately 1.00 inches. In some embodiments, the fourth grip segment 914 may have a length of approximately 4.055 inches and a width of approximately 0.787 inches.

In some embodiments, the grip segments 902, 904, 906, and 914 may each include a plurality of perforations. The perforations 908 may have a diameter of approximately 0.10 inches and may be arranged linearly along a longitudinal direction of the grip segment. The perforations may be spaced apart at a pitch of approximately 0.25 inches to approximately 0.30 inches. In some embodiments, the perforations 908 may be spaced apart at a pitch of approximately 0.28 inches. In some embodiments, the first grip segment 902 may include four perforations, the second and third grip segments 904, 906 may include six grip segments, and the fourth grip segment may include fifteen perforations. It should be noted that the dimensions and spacing of the grip segments and perforations described with respect to FIGS. 13A-13B may be included in any of the embodiments described herein.

The grip segments may be arranged on the layer of repair fabric to cover a percentage of the body portion surface to maximize tissue grip. In some embodiments, the grip segments may be arranged on the body portion to cover greater than or equal to approximately 10%, 15%, 20%, or 25% of the surface area of the body portion 100. In some embodiments, the grip segments may be arranged on the body portion to cover less than or equal to approximately 45%, 40%, 35%, 30%, or 25% of the surface area of all sizes of body portion 100. Any suitable combinations of the above-referenced ranges are also possible. In one embodiment, the grip segments may be arranged on the body portion to cover greater than or equal to approximately 20% and less than or equal to 30% of the surface area of the body portion.

In some embodiments, the grip segments may be made of polylactide acid (PLA), but the disclosure is not so limited, and the grip segments may be made of other suitable resorbable materials (e.g., P4HB, etc.). In some embodiments, a prosthesis may include four grip segments (see FIGS. 13A and 13B). In such embodiments, the total surface area of the grip segments on the prosthesis may be approximately 5.3 square inches to approximately 7.6 square inches, depending on the size of the prosthesis. The total surface area of the exposed layer of repair fabric of the body portion may be approximately 17.5 square inches to approximately 28.4 square inches, depending on size of the prosthesis. In some embodiments, the grip segments may occupy approximately 26% to approximately 30.2% of the surface area of the body portion.

It should be appreciated that all grip segments, perforations, and patterns described in each embodiment are non-limiting examples and any configuration may be used as needed in practice. It should also be noted that any grip segment and configuration from each embodiment described above may be used in any prosthesis.

While several embodiments of the present invention have been described and illustrated herein, those of ordinary skill in the art will readily envision a variety of other features and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. An implantable prosthesis comprising:
a layer of biologically compatible repair fabric; and
a plurality of grip segments integrated with the layer of repair fabric, each of the plurality of grip segments includes a plurality of grips and a plurality of perforations extending therethrough.

2. The implantable prosthesis of claim 1, wherein the plurality of grip segments includes a plurality of microtextured grip segments.

3. The implantable prosthesis of claim 1, wherein each of the plurality of grips includes a first pillar and a plurality of second pillars extending from a surface of the first pillar.

4. The implantable prosthesis of claim 1, wherein the plurality of grip segments includes three or more grip segments integrated with the layer of repair fabric.

5. The implantable prosthesis of claim 1, wherein at least one of the plurality of grip segments has an L-shaped configuration.

6. The implantable prosthesis of claim 1, wherein at least one of the plurality of grip segments has an elliptical shaped configuration.

7. The implantable prosthesis of claim 1, wherein the plurality of grip segments are arranged on the layer of repair fabric to maintain a center portion of the repair fabric free of grip segments.

8. The implantable prosthesis of claim 1, wherein the layer of repair fabric has a preformed three-dimensional configuration with an apex which is free of grip segments.

9. The implantable prosthesis of claim 1, wherein the plurality of perforations on at least one of the plurality of grip segments occupy approximately 6% to approximately 20% of the grip segment.

10. The implantable prosthesis of claim 1, wherein the plurality of grip segments occupy approximately 20% to approximately 30% of a surface of the layer of repair fabric.

11. The implantable prosthesis of claim 1, wherein each of the plurality of perforations has a diameter of approximately 0.100 inches to approximately 0.110 inches.

12. The implantable prosthesis of claim 1, wherein the plurality of perforations are spaced apart at a center distance of approximately 0.25 inches to approximately 0.30 inches.

13. The implantable prosthesis of claim 1, wherein the plurality of perforations for at least one of the plurality of grip segments are arranged in at least three distinct regions on the grip segment.

14. The implantable prosthesis of claim 13, wherein each region includes a group of perforations.

15. The implantable prosthesis of claim 13, wherein a distance between regions is greater than a distance between adjacent perforations in a region.

16. The implantable prosthesis of claim 14, wherein perforations of each group have uniform spacing within each region.

17. The implantable prosthesis of claim 1, wherein the plurality of perforations for at least one of the plurality of grip segments are arranged in a two-dimensional pattern with equal spacing.

18. The implantable prosthesis of claim 1, wherein the plurality of perforations for at least one of the plurality of grip segments are arranged in an array which extends in a direction along a length of the grip segment.

19. The implantable prosthesis of claim 1, wherein the plurality of perforations for at least one of the plurality of grip segments are arranged in a hexagonal pattern.

20. The implantable prosthesis of claim 1, wherein the plurality of grips are arranged in a hexagonal pattern on at least one of the grip segments.

21. The implantable prosthesis of claim 1, wherein the layer of repair fabric has a preformed three-dimensional configuration with an apex and a rounded ridge extending from the apex to an outer periphery of the layer of repair fabric, a portion of at least one of the plurality of grip segments extending across the rounded ridge.

22. The implantable prosthesis of claim 1, wherein each of the plurality of grip segments includes a substrate with opposing first and second surfaces, at least one of the plurality of grip segments including a plurality of grips on each of the first and second surfaces, the plurality of grips on the first surface facing away from the layer of repair fabric and the plurality of grips on the second surface facing toward the layer of repair fabric.

23. The implantable prosthesis of claim 21, wherein at least two grip segments are arranged on the layer of repair fabric entirely on one side of the rounded ridge.

24. The implantable prosthesis of claim 23, wherein the at least two grip segments have elliptical shaped configurations, and wherein the at least two grip segments are positioned with a long-side of the grip segment approximately parallel to a portion of a periphery of the layer of repair fabric.

25. The implantable prosthesis of claim 21, wherein at least one grip segment is arranged on the layer of repair fabric closer to a periphery of the grip segment than to the rounded ridge.

26. The implantable prosthesis of claim 1, wherein the plurality of grip segments includes six or more grip segments integrated with the layer of repair fabric.

27. An implantable prosthesis comprising:
a layer of biologically compatible repair fabric; and
a plurality of microtextured grip segments integrated with the layer of repair fabric, each of the plurality of microtextured grip segments includes a plurality of perforations extending therethrough.

28. The implantable prosthesis of claim 27, wherein the plurality of microtextured grip segments includes three or more grip segments integrated with the layer of repair fabric.

29. The implantable prosthesis of claim 27, wherein at least one of the plurality of microtextured grip segments has an L-shaped configuration.

30. The implantable prosthesis of claim 27, wherein at least one of the plurality of microtextured grip segments has an elliptical shaped configuration.

31. The implantable prosthesis of claim 27, wherein the plurality of microtextured grip segments are arranged on the layer of repair fabric to maintain a center portion of the repair fabric free of microtextured grip segments.

32. The implantable prosthesis of claim 27, wherein the layer of repair fabric has a preformed three-dimensional configuration with an apex which is free of microtextured grip segments.

33. The implantable prosthesis of claim 27, wherein the plurality of perforations on at least one of the plurality of microtextured grip segments occupy between approximately 6% to 20% of the microtextured grip segment.

34. The implantable prosthesis of claim 27, wherein the plurality of microtextured grip segments occupy approximately 20% to approximately 30% of a surface of the layer of repair fabric.

35. The implantable prosthesis of claim 27, wherein each of the plurality of perforations has a diameter of approximately 0.100 inches to approximately 0.110 inches.

36. The implantable prosthesis of claim 27, wherein the plurality of perforations are spaced apart at a center distance of approximately 0.25 inches to approximately 0.30 inches.

37. The implantable prosthesis of claim 27, wherein the plurality of perforations for at least one of the plurality of microtextured grip segments are arranged in at least three distinct regions on the microtextured grip segment.

38. The implantable prosthesis of claim 27, wherein the plurality of perforations for at least one of the plurality of microtextured grip segments are arranged in a two-dimensional pattern with equal spacing.

39. The implantable prosthesis of claim 27, wherein plurality of perforations for at least one of the plurality of microtextured grip segments are arranged in an array which extends in a direction along a length of the microtextured grip segment.

40. The implantable prosthesis of claim 27, wherein the plurality of perforations for at least one of the plurality of microtextured segments are arranged in a hexagonal pattern.

41. A method of fabricating an implantable prosthesis, the method comprising:
providing a layer of biologically compatible repair fabric; and
attaching a plurality of grip segments to a surface of the layer of repair fabric, wherein each of the grip segments includes a plurality of grips and a plurality of perforations extending therethrough.

42. The method of claim 41, wherein the plurality of grip segments occupy approximately 20% to approximately 30% of a surface of the layer of repair fabric.

43. The method of claim 41, further comprising arranging the plurality of grip segments on the layer of repair fabric to maintain a center portion of the repair fabric free of grip segments.

44. The method of claim 41, wherein the perforations occupy approximately 6% to approximately 20% of the grip segment.

45. The method of claim 41, wherein each of the plurality of perforations has a diameter of approximately 0.100 inches to approximately 0.110 inches.

46. The method of claim 41, wherein the plurality of perforations are spaced apart at a center distance of approximately 0.25 inches to approximately 0.30 inches.

47. The method of claim 41, wherein the plurality of perforations for at least one of the plurality of grip segments are arranged in at least three distinct regions on the grip segment.

48. The method of claim 41, wherein the plurality of perforations for at least one of the plurality of grip segments are arranged in a two-dimensional pattern with equal spacing.

49. The method of claim 41, wherein the plurality of perforations for at least one of the plurality of grip segments are arranged in an array which extends in a direction along a length of the grip segment.

50. The method of claim 41, wherein the plurality of perforations for at least one of the plurality of grip segments are arranged in a hexagonal pattern.

51. An implantable prosthesis comprising:
a layer of biologically compatible repair fabric having a preformed three-dimensional configuration with an apex and a rounded ridge extending from the apex to an outer periphery of the layer of repair fabric; and
a plurality of grip segments integrated with the layer of biologically compatible repair fabric, wherein a portion of at least one of the plurality of grip segments extends across the rounded ridge.

52. The implantable prosthesis of claim 51, wherein at least one of the plurality of grip segments has an L-shaped configuration.

53. The implantable prosthesis of claim 51, wherein at least one of the plurality of grip segments has an elliptical shaped configuration.

54. The implantable prosthesis of claim 51, wherein the plurality of grip segments are arranged on the layer of repair fabric to maintain a center portion of the repair fabric free of grip segments.

55. The implantable prosthesis of claim 51, wherein the apex is free of grip segments.

56. The implantable prosthesis of claim 51, wherein the plurality of grip segments occupy approximately 20% to approximately 30% of a surface of the layer of repair fabric.

57. The implantable prosthesis of claim 51, wherein at least two grip segments are arranged on the layer of repair fabric entirely on one side of the rounded ridge.

58. The implantable prosthesis of claim 51, wherein the at least two grip segments have elliptical shaped configurations, and wherein the at least two grip segments are positioned with a long-side of each grip segment extending in a direction corresponding to a portion of a periphery of the layer of repair fabric.

59. The implantable prosthesis of claim 51, wherein at least one grip segment is arranged on the layer of repair fabric closer to a periphery of the grip segment than to the rounded ridge.

60. The implantable prosthesis of claim 51, wherein the plurality of grip segments includes six or more grip segments attached to the layer of repair fabric.

61. An implantable prosthesis comprising:
a layer of biologically compatible repair fabric; and
a plurality of grip segments integrated with the layer of repair fabric, each of the plurality of grip segments includes a substrate and a plurality of grips extending from a surface of the substrate, wherein each of the plurality of grips includes a first pillar and a plurality of second pillars extending from a surface of the first pillar.

62. The implantable prosthesis of claim 61, wherein each of the plurality of grip segments includes a plurality of perforations extending therethrough.

63. The implantable prosthesis of claim 61, wherein the plurality of grip segments includes three or more grip segments integrated with the layer of repair fabric.

64. The implantable prosthesis of claim 61, wherein at least one of the plurality of grip segments has an L-shaped configuration.

65. The implantable prosthesis of claim 61, wherein at least one of the plurality of grip segments has an elliptical shaped configuration.

66. The implantable prosthesis of claim 61, wherein the plurality of grip segments are arranged on the layer of repair fabric to maintain a center portion of the repair fabric free of grip segments.

67. The implantable prosthesis of claim 61, wherein the layer of repair fabric has a preformed three-dimensional configuration with an apex which is free of grip segments.

68. The implantable prosthesis of claim 61, wherein the plurality of grip segments occupy approximately 20% to approximately 30% of a surface of the layer of repair fabric.

69. The implantable prosthesis of claim 61, wherein the plurality of grips are arranged in a hexagonal pattern on at least one of the grip segments.

70. A grip segment for an implantable prosthesis, the grip segment comprising:
a substrate of biologically compatible material attachable to a layer of repair fabric;
a plurality of microstructure grips extending from a surface of the substrate, wherein each of the plurality of microstructure grips comprises a first pillar and a plurality of second pillars extending from a first surface of the first pillar; and
a plurality of perforations extending through the substrate.

71. The grip segment of claim 70, wherein the grip segment has an L-shaped configuration.

72. The grip segment of claim 70, wherein the grip segment has an elliptical shaped configuration.

73. The grip segment of claim 70 wherein the plurality of perforations on at least one of the plurality of grip segments occupy approximately 6% to approximately 20% of the grip segment.

74. The grip segment of claim 70, wherein each of the plurality of perforations has a diameter of approximately 0.100 inches to approximately 0.110 inches.

75. The grip segment of claim 70, wherein the plurality of perforations are spaced apart at a center distance of approximately 0.25 inches to approximately 0.30 inches.

76. The grip segment of claim 70, wherein the plurality of perforations are arranged in three distinct regions on the grip segment.

77. The grip segment of claim 70, the plurality of perforations are arranged in a two-dimensional pattern with equal spacing.

78. The grip segment of claim 70, wherein the plurality of perforations are arranged in an array which extends in a direction along a length of the grip segment.

79. The grip segment of claim 70, wherein the plurality of perforations are arranged in a hexagonal pattern.

80. The grip segment of claim 70, wherein the plurality of grips are arranged in a hexagonal pattern on a grip segment.

81. The grip segment of claim 70, wherein each of the plurality of second pillars extends from the first surface of the first pillar in a substantially perpendicular direction.

82. The grip segment of claim 70, wherein the first pillar has a diameter of approximately 0.09 inches to approximately 0.11 inches.

83. The grip segment of claim 70, wherein the first pillar has a height of approximately 0.045 inches to approximately 0.055 inches.

84. The grip segment of claim 70, wherein the plurality of microstructure grips are spaced apart in a row at a pitch of approximately 0.18 inches to approximately 0.22 inches.

85. The grip segment of claim 70, wherein each of the plurality of second pillars has a diameter of approximately 0.009 inches to approximately 0.011 inches.

86. The grip segment of claim 70, wherein each of the plurality of second pillars has a height of approximately 0.018 inches to approximately 0.022 inches.

87. The grip segment of claim 70, wherein each of the plurality of second pillars are spaced apart in a row at a pitch of approximately 0.018 inches to approximately 0.022 inches.

88. The grip segment of claim 70, wherein the first pillar has a cylindrical shape and has a shaft angle relative to the surface of the substrate of approximately 3 degree to approximately 7 degrees.
